(19) United States
(12) Patent Application Publication
(10) Pub. No.: US 2021/0082053 A1
(43) Pub. Date: Mar. 18, 2021

(54) SYSTEM FOR DETERMINING MEDICATION COVERAGE BY INSURANCE CARRIERS

(71) Applicants: Michele Street, Lake Stevens, WA (US); Melanie L. Harper, Snohomish, WA (US)

(72) Inventors: Michele Street, Lake Stevens, WA (US); Melanie L. Harper, Snohomish, WA (US)

(21) Appl. No.: 16/568,806

(22) Filed: Sep. 12, 2019

(51) Int. Cl. G06Q 40/08; G16H 40/00; G16H 20/10

(52) U.S. Cl. CPC G06Q 40/08; G16H 20/10; G16H 40/00

(57) ABSTRACT

The system includes a sequence of selections in a processing system, including a geographical state, the patient's insurance carrier operating within the state, the plan type of the insurance carrier for the patient and a search criteria for one or more medications selected by a prescriber for medical treatment of the patient. The processor provides insurance coverage status information for medications meeting the search criteria obtained from the insurance carrier plan type formulary.

### TECHNICAL FIELD

This invention relates generally to determining medication coverage for medical treatment of individual patients and more specifically concerns such determination based on a sequence of separate information selections for the patient.

### BACKGROUND OF THE INVENTION

Effective treatment for a patient's medical condition often involves prescribing one or more medications, typically paid for by the patient's insurance plan. The prescribed medications will vary depending upon the particular medical condition being treated and may or may not be covered by the patient's insurance plan. When a medication is prescribed for a specific medical treatment, it must be determined whether the particular medication is covered or not covered by the patient's insurance plan, the co-pay required or if the medication requires a prior authorization. Often this results in a significant time gap between the time of the prescription and the delivery of the prescribed medication to the patient. In some cases, the medication will be covered and the patient can proceed to the pharmacy. In other cases, because of increased co-pay or if prior authorization is required, the prescriber (physician or other authorized provider) may select another drug having better coverage or proceed with a prior authorization request of the insurance carrier. This process can result in a delay of the patient in obtaining a prescription, potentially leading to adverse health consequences.

Accordingly, the present invention is a system for providing fast and accurate determination of medication coverage, typically when the patient is with the prescriber, improving the delivery time of the medication to the patient for effective treatment of a medical condition.

### SUMMARY OF THE INVENTION

Accordingly, a system is disclosed for determining insurance coverage status of prescription drugs for medical treatment of patients, comprising: a processing system providing a sequence of selections for a medication prescribing person, including: a first selection for a geographical state of a patient to identify insurance carriers providing prescription medications in the selected state; a second selection of an insurance carrier of the patient operating in the selected state; a third selection of a plan type of the patient from the selected insurance carrier; a fourth selection of a medical search criteria for at least one of the following: specific organ system, specific disease state, specific drug name and specific drug class, wherein the fourth selection will include a list satisfying the selected search criteria; and a fifth selection of one from said list satisfying the selected search criteria; wherein; the processing system in operation uses the second, third, fourth and fifth selections to determine insurance coverage status for medication or medications selected by the prescriber for medical treatment of the patient.

### BRIEF DESCRIPTION OF THE DRAWINGS

FIG. 1 is a diagram illustrating the medication determining system of the present invention.

### BEST MODE FOR CARRYING OUT THE INVENTION

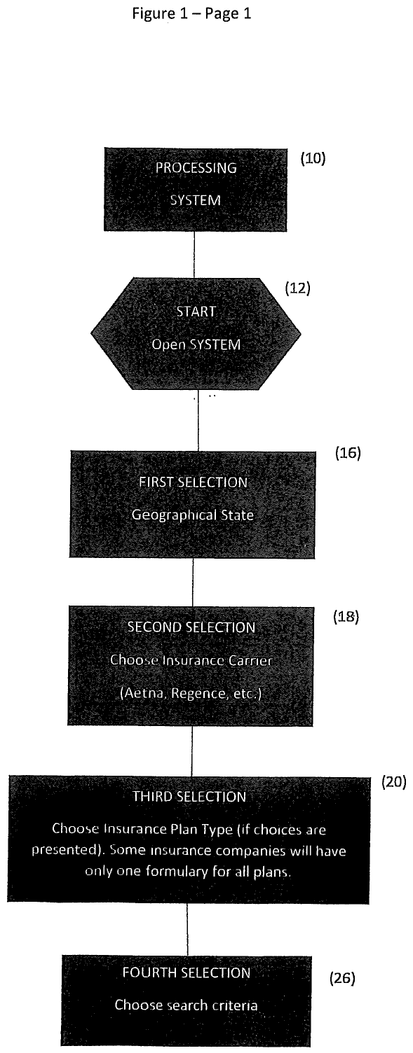

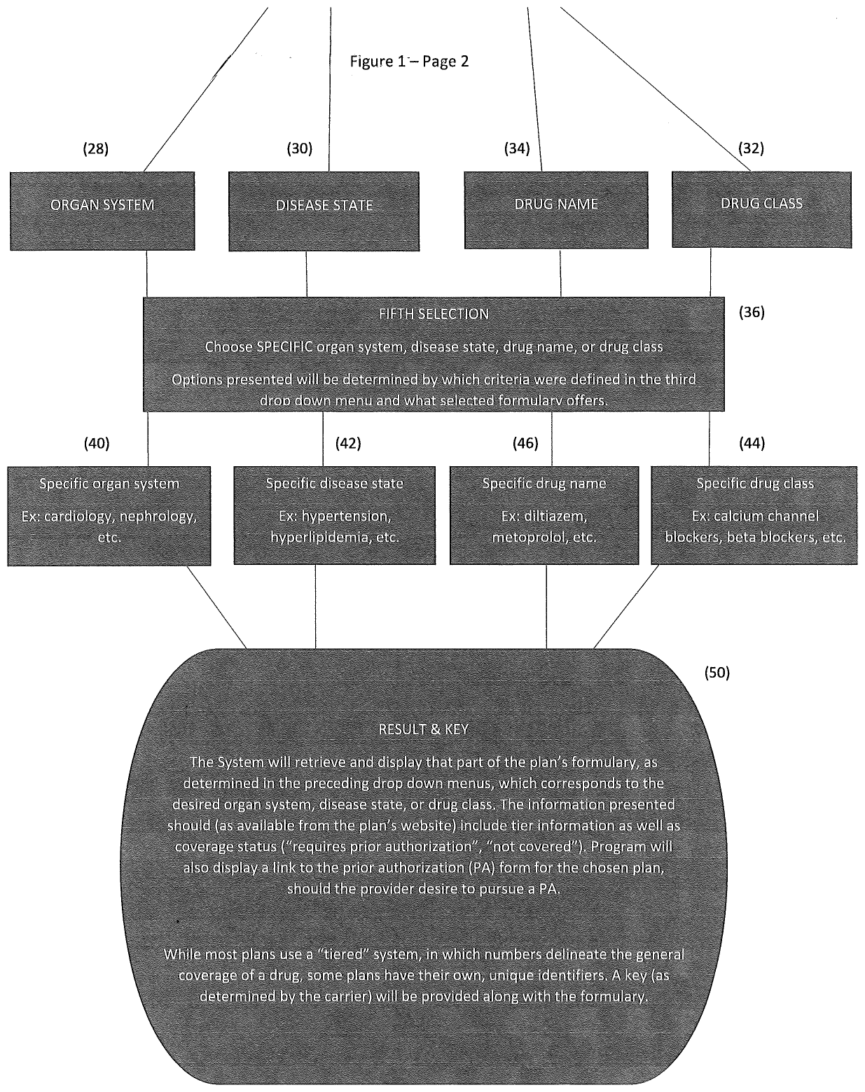

Referring to FIG. 1, the system of the present invention includes a first operating selection or step, in particular opening up the system 12 for use by a physician or other prescriber/provider shown generally at 12. The system is under the control of a processing system 10. In a second selection, the prescriber is presented with selecting or choosing a particular geographic state, e.g. Alaska, etc. from a stored source shown at 16. The selected state will typically be the residence of the patient. After a state is selected, the insurance carrier or other medication plan provider, such as a company employer of the patient, is identified. A list of insurance carriers for each state is provided at 18. Examples of insurance carriers include Aetna, Regence, Blue Shield etc., these are examples only; all insurance carriers for the selected state will be presented for selection by the prescriber. The prescriber then selects the insurance carrier of the patient he/she is serving. The insurance carrier will be known to the prescriber by a patient's insurance card or other source of information. The system then allows the prescriber to select the particular insurance plan type of the patient, shown at 20. This information is provided as well by the patient's insurance card or other source of insurance information. The insurance plan type will typically be identified by a plan code. The insurance plan code could be a number, a word or a combination thereof, this will depend on the insurance carrier. Each insurance carrier will have a number of different plan types available, with different coverages, costs and co-pays. In some cases there may be only one plan type. In some cases, each plan type will have its own formulary of prescription drugs. In some cases all plan types will have the same formulary. Again, the above information is conveniently available to the prescriber by an insurance card or other available source. After the insurance plan type has been identified, medication treatment search criteria are identified in order to access prescription drug formulary information, shown at 26. Typically, search criteria include four options; 1: organ system 28; 2: disease state 30; 3: drug class 32; 4: drug name 34. Selection of a search criteria from the above is identified as a fourth selection. Generally, the two search criteria most often selected are drug class and drug name. In many cases, the prescriber will simply select a drug name to search. At 36, the search criteria 26 is made more specific in a fifth selection, as discussed below. Depending upon the particular search criteria selected, and the fifth selection, drug information is provided which satisfies the search criteria and the fifth selection from the preselected formulary of prescription medications available, typically involving 100-200 pages of individual drugs.

For example, a specific organ system selection, shown at 40, will result in a list of medications associated with a selected specific organ system, for example, the illustration: cardiovascular, digestive (GI), endocrine, immune, integumentary, lymphatic, muscular, nervous, reproductive, respiratory, skeletal, and urinary. Other specific organ system lists can be used. Selection of one of the specific organ systems results in a list of medications which are associated with the selected specific organ system, as explained in further detail below.

The second search criterion under fifth selection 36 noted above is for a specific disease state, shown at 42. An example of a list of disease states is as follows: AIDS (acquired immune deficiency syndrome); asthma; arthritis; bacterial meningitis; blood disorders; breast cancer; bronchitis; cancer; chlamydia; chronic kidney disease; common cold; concussion; deep vein thrombosis; dengue fever; depression; diabetes; epilepsy; Epstien-Barr virus infection; fibromyalgia; food-borne illness; gout; hand, foot and mouth disease; head lice; hemophilia; herpes zoster; hypertension (high blood pressure); hyperlipidemia (high cholesterol); influenza; jaundice; kawasaki disease; listeriosis; lupus; malaria; measles; meningitis; norovirus infection; obesity; otitis media; peripheral artery disease; pertussis (whooping cough); pneumococcal disease; polio; rabies; ring worm; rubella; scabies; septicemia; sickle cell disease; tuberculosis; Tourette's syndrome; trichinosis; typhoid fever; ulcerative colitis; uterine cancer; varicella disease (chicken pox); Von Willebrand disease; West Nile virus infection; yellow fever; zika virus infection. Again, this is an example of a list in the present system relative to specific disease states. Other specific disease state lists can be used.

A third search criterion shown at 44 is a specific drug class. An example of an insurance carrier plan type formulary of drug classes is as follows.

### Analgesics

analgesics, other
nsaids
nsaids, topical
cox-2 inhibitors
gout
opioid analgesics
non-opioid analgesics

### Anti-Infectives

antibacterials
antifungals
antimalarials
antiretroviral agents
antitubercular agents
antivirals
miscellaneous

### Antineoplastic Agents

alkylating agents
antimetabolites
cytoprotective agents
hormonal antineoplastic agentsimmunomodulators
kinase inhibitors
miscellaneous

### Cardiovascular

ace inhibitors
ace inhibitor/calcium channel blocker combinations
ace inhibitor/diuretic combinations
adrenolytics, central
aldosterone receptor antagonists
alpha blockers
angiotensin ii receptor antagonists/diuretic combinations
angiotensin ii receptor antagonist/calcium channel blocker combinations
antiarrhythmics
antilipemics
beta-blockers
beta-blocker/diuretic combinations
calcium channel blockers
digitalis glycosides
diuretics
heart failure
nitrates
pulmonary arterial hypertension
vasopressors
miscellaneous

### Central Nervous System

antianxiety
anticonvulsants
antidementia
antidepressants
antiparkinsonian agents
antipsychotics
attention deficit hyperactivity disorder
huntington's disease agents
hypnotics
migraine
mood stabilizers
multiple sclerosis agents
musculoskeletal therapy agents
myasthenia gravis
narcolepsy/cataplexy
psychotherapeutic-miscellaneous
miscellaneous

### Endocrine and Metabolic

acromegaly
androgens
antidiabetics
calcium receptor antagonists
calcium regulators
carnitine deficiency agents
contraceptives
endometriosis
estrogens
estrogen/progestins
estrogen/selective estrogen receptor modulator combinations
gaucher disease
glucocorticoids
glucose elevating agents
human growth hormones
hyperparathyroid treatment, vitamin d analogs
insulin-like growth factors
phosphate binder agents
potassium-removing agents
progestins
selective estrogen receptor modulators
thyroid agents
vasopressins
vasopressin receptor antagonists
miscellaneous

### Gastrointestinal

antacids
antidiarrheals
antiemetics
antispasmodics
cholelitholytics
h2 receptor antagonists
inflammatory bowel disease irritable bowel syndrome
laxatives/stool softeners
opioid-induced constipation
pancreatic enzymes
prostaglandins
proton pump inhibitors
saliva stimulants
steroids, rectal
ulcer therapy combinations miscellaneous

### Genitourinary

benign prostatic hyperplasia
urinary antispasmodics
vaginal anti-infectives
miscellaneous

### Hematologic

anticoagulants
hematopoietic growth factors
hemophilia a agents
hemophilia b agents
hereditary angioedema agents
iron chelating agents
paroxysmal nocturnal hemoglobinuria (pnh) agents
platelet aggregation inhibitors
platelet synthesis inhibitors
thrombocytopenia agents
miscellaneous

### Immunologic Agents

allergenic extracts
autoimmune agents
disease-modifying antirheumatic drugs (dmards)
immune globulins
immunomodulators
immunosuppressants
vaccines
miscellaneous

### Nutritional/Supplements

electrolytes
vitamins and minerals

### Respiratory

alpha-1 antitrypsin deficiency agents anaphylaxis treatment agents
anticholinergics
anticholinergic/beta agonist combinations
antihistamines
beta agonists
cough and cold
cystic fibrosis
leukotriene modifiers
mast cell stabilizers
medical supplies
nasal antihistamines
nasal decongestants
nasal steroids
phosphodiesterase-4 inhibitors
pulmonary fibrosis agents
respiratory syncytial virus
severe asthma agents
steroid/beta agonist combinations
steroid inhalants
xanthines
miscellaneous

### Topical

dermatology
mouth/throat/dental agents
ophthalmic
otic

### Miscellaneous

irrigation solutions
medical supplies
   Again, this specific drug class list, which is frequently used as a search criteria by prescribers, can be changed in various specific embodiments.

A fourth category 46, is a formulary list of specific drug names which may be individually selected by the prescriber for treatment of the patient's medical condition. The purpose of this fourth search criteria is to identify for the prescriber a particular drug name or drug names which are available in the formulary.

The system will use the selection to identify, in box 50, i.e. retrieve, the coverage status and that part of the selected plan's formulary of medications which meet the selected search criteria. The result will be coverage information for drugs meeting the search criteria in the form of a PDF available to the prescriber, from which the prescriber and the patient can choose. The actual specific drug(s) are listed line by line in the formulary, with a coverage information indication, i.e. tier information, attached. Typically, as the tiers increase, the patient pays more.

Generally, by way of additional back ground information for the present invention, drugs are classified (tiered) based on the drug; most insurance carriers will have one preferred drug in each tier. The actual drug name or brand name is used and the class assigned is in the formulary. Some of the key examples include: covered; not covered; preferred generic; generic; preferred brand; non-preferred brand, specialty drug or prior authorization. There will exist a link below the PDF box that will allow the provider to retrieve, open and print, the prior authorization request form from the insurance company, which the provider can complete in order to begin the request for prior authorization. Each insurance company will have its own formulary and a key which identifies the characteristic codes for the formulary, which may include examples like specific tier, quantity limits, prior authorization required, age, specialty step therapy, etc. The prescriber can then identify the particular drug to be prescribed and will provide a prescription, electronic or written, for that drug. There is thus no lost time relative to identifying the drug for medical treatment which fits the patients' insurance plan. This includes proceeding immediately with the prior authorization process, if necessary or specialty drug authorization.

All this can be quickly done in the present system with the patient present with the prescriber. A prescription can be written from the PDF which the patient can then fill without any lost time from communication with the insurance carrier related to drug coverage thereof, decreasing barriers to covered selected drugs, decreasing confusion relative to coverage and improving delivery time to the patient. A significant advantage to the present system is its simplicity and convenience for the provider.

Although a preferred embodiment of the invention has been disclosed for purposes of illustration, it should be understood that various changes, modifications and substitutions may be incorporated in the embodiment without departing from the spirit of the invention, which is defined by the claims which follow.

What is claimed is:

1. A system for determining insurance coverage status of prescription drugs for medical treatment of patients, comprising:
a processing system providing a sequence of selections for a medication prescribing person, including:
a first selection for a geographical state of a patient to identify insurance carriers providing prescription medications in the selected state;
a second selection of an insurance carrier of the patient operating in the selected state;
a third selection of a plan type of the patient from the selected insurance carrier;
a fourth selection of a medical search criteria for at least one of the following: specific organ system, specific disease state, specific drug name and specific drug class, wherein the fourth selection will include a list satisfying the selected search criteria; and
a fifth selection of one from said list satisfying the selected search criteria; wherein; the processing system in operation uses the second, third, fourth and fifth selections to determine insurance coverage status for medication or medications selected by the prescriber for medical treatment of the patient.

2. The system of claim 1, wherein the insurance coverage status includes medications covered, medications not covered and prior authorization required.

3. The system of claim 1, wherein the results of the processing system are in the form of a pdf. display of the insurance carrier plan type formularies.

4. The system of claim 1, wherein the selection steps are made in sequence.
